(19) United States
(12) Patent Application Publication
(10) Pub. No.: US 2022/0142964 A1
(43) Pub. Date: May 12, 2022

(54) PHARMACEUTICAL DOSAGE FORM OF COLESTIPOL

(71) Applicant: Edenbridge Pharmaceuticals, LLC, Parsippany, NJ (US)

(72) Inventors: Venkatesh Naini, Parsippany, NJ (US); Velayutham Kanniyappan, Monmouth Junction, NJ (US)

(73) Assignee: Edenbridge Pharmaceuticals, LLC, Parsippany, NJ (US)

(21) Appl. No.: 17/093,775

(22) Filed: Nov. 10, 2020

(51) Int. Cl. A61K 31/336; A61K 9/16; A61K 31/132

(52) U.S. Cl. CPC A61K 31/336; A61K 9/1694; A61K 9/1635; A61K 9/1652; A61K 31/132; A61K 9/1617; A61K 9/1641; A61K 9/1664; A61K 9/1611

(57) ABSTRACT

The present invention contemplates a high dose finished pharmaceutical dosage form comprising colestipol hydrochloride in its commercially available form (i.e., beads) without the need for further milling. A manufacturing process used to manufacture the instant high dose finished pharmaceutical dosage form comprising colestipol hydrochloride, which monitors moisture content throughout the granulation process, is also disclosed herein.

### BACKGROUND OF THE INVENTION

Colestipol hydrochloride is an antihyperlipidemic agent used to decrease serum cholesterol levels. It is an insoluble, high molecular weight anion-exchange co-polymer that binds bile acids in the intestine (bile acid sequestrant), forming a complex, which is then excreted in the feces. This allows the removal of bile acids from circulation, which otherwise aid in breakdown of fats in dietary lipids and their subsequent absorption into systemic circulation. This local activity in the gut and its non-absorption into systemic circulation, makes it an attractive therapy for use in patients who cannot tolerate systemically acting antihyperlipidemic agents such as statins and fibrates. Colestid tablets and Colestid® granules for oral suspension and their associated generic equivalents are the only colestipol hydrochloride products approved by the United States Food and Drug Administration (“USFDA”) for use in humans. Other bile acid sequestrants, colesevelam hydrochloride and cholestyramine, are also approved by USFDA.

Locally-acting polymeric drugs such as colestipol hydrochloride present formulation and processing challenges to a pharmaceutical formulation scientist because of the high doses required to be efficacious. For example, Colestid® tablets are available in only one strength, 1,000 mg/1 g, and recommended doses range from 2 to 16 grams per day (given once or in divided doses). The term “high dose” as used herein is defined to mean 70% w/w or more of API in a single unit of a dosage form (e.g., a tablet). High dose finished pharmaceutical dosage forms are a challenge to a pharmaceutical formulation scientist because the lack of excipient(s) makes it difficult to get suitable compression. As used herein, a “finished” pharmaceutical dosage form is one that has completed pharmaceutical manufacturing, including any required coating steps. Further, as is the case with colestipol hydrochloride, the active pharmaceutical ingredient (“API”) may not have suitable compaction properties. Not only are high dose dosage forms (e.g., tablets) a challenge for a pharmaceutical formulation scientist, they are also a challenge for patients who must swallow the high dose dosage form. For example, Colestid® tablets are approximately 18.7 mm (length)×9.9 mm (width)×9.7 mm (height/thickness) in size and approximately 1150 mg (1134-1166 mg in representative samples) in total weight, which represents a significant challenge for a human patient who must swallow the tablets. The only FDA-approved generic equivalent if Colestid® tablets, which was approved before the implementation of Guidance for Industry: Size, Shape, and Other Physical Attributes of Generic Tablets and Capsules in June 2015 is even larger in size than Colestid® tablets at approximately 21.1 mm (length)×9.8 mm (width)×10.4 mm (height/thickness) in size and 1550 mg (1547-1571 mg in representative samples) in weight. There is thus a need for a compact dosage, such as a tablet, which can incorporate same dose of colestipol hydrochloride, into a smaller, more elegant dosage form to enhance swallowability. The Colestid® granules for oral suspension, which have a gritty texture, also present compliance challenges for patients.

Chemically, colestipol hydrochloride is a high molecular weight co-polymer of diethylenetriamine and 1-chloro-2,3-epoxypropane (hydrochloride), with approximately 1 out of 4 amine nitrogen groups being protonated. It is a light-yellow insoluble resin. It is hygroscopic and swells when it comes in contact with water or aqueous fluids.

The hydroscopic nature of colestipol hydrochloride, which causes it to swell when it comes in contact with water or aqueous fluids, also causes a significant problem for a pharmaceutical formulation scientist. Water or aqueous fluids in the finished pharmaceutical dosage form (i.e., the dosage form after all pharmaceutical processing steps) can present stability issues for the product, which result in a lower shelf life. For example, water or aqueous fluids can cause physical instability (e.g., swelling and loss of dosage form integrity). Processing for a dosage form of colestipol hydrochloride is also a challenge because the API wants to absorb water or aqueous fluids whenever available; therefore, a pharmaceutical formulation scientist must limit exposure of the API to water or other aqueous fluids. This is a significant problem for a pharmaceutical formulation scientist because the pharmaceutical processing techniques required to densify the dosage form (densification is necessary to reduce the size of the dosage form) often rely on water or aqueous fluids. These same processing techniques improve tablet compression. Even for dry processes a higher moisture content of the final blend before compression (either API by itself or API with excipients) improves the compaction properties of the final blend. There is thus a delicate balance between having enough water and/or aqueous fluids to yield a suitable dosage form (e.g., a tablet) and protecting the dosage form from degradation and attaining suitable stability of the dosage form.

The drug substance for colestipol hydrochloride is typically produced by a polymerization process, known in the art as the “bead process” as described in U.S. Pat. No. 3,803,237. As depicted in FIG. 1A, the drug substance used in the Colestid® granules for oral suspension product is these “beads,” which are spherical in nature. In order to reduce the size of a dosage form comprising colestipol hydrochloride drug substance produced by the bead process, the particle size must be further reduced or micronized. U.S. Pat. No. 5,520,932—which describes the use of an expensive, complex and labor-intensive process, whereby a cutting mill is used to reduce the colestipol spherical particles to non-spherical shape—specifically describes the inability of conventional pharmaceutical mills to reduce the particle size of colestipol beads. Colestid® tablets are made using the process disclosed in U.S. Pat. No. 5,520,932, which is depicted in FIG. 1B showing the particles derived from Colestid tablets.

### BRIEF SUMMARY OF THE INVENTION

The present invention contemplates a high dose finished pharmaceutical dosage form comprising colestipol hydrochloride in its commercially available form (i.e., beads) without the need for further milling. The present invention may optionally include one or more pharmaceutically acceptable excipients. The present invention identifies the correct balance between the need to reduce wetting and swelling of colestipol particles during manufacturing with the need to achieve granules with a suitable moisture content for tableting. The high dose finished pharmaceutical dosage form of the instant invention attains (i) acceptable pharmaceutical stability, which includes maintaining physical and chemical integrity over shelf life and (ii) in vitro and in vivo bile acid binding performance similar to Colestid.

The high dose finished pharmaceutical dosage form of the instant invention is made using a granulation process. A novel approach is described to monitor the moisture content of granules throughout the entire granulation process. The process described does not require any further manipulation or reduction of the Colestipol particle size and uses conventional pharmaceutical equipment, with shorter processing times as compared to traditional wet granulation using high-shear or fluid bed processing; thus, the present invention does not require the use of the cutting mill described in U.S. Pat. No. 5,520,932. The manufacturing process and techniques employed herein may be applicable to other high dose, insoluble polymeric drug substances such as, without limitation cholestyramine, colextran, colestilan, colesevelam, sevelemer hydrochloride, sevelemer carbonate, sodium polystyrene sulfonate, and patiromer.

### BRIEF DESCRIPTION OF THE DRAWINGS

FIG. 1. Microscopic images of colestipol particles: (FIG. 1A) API from Colestid® granules for oral suspension; (FIG. 1B) API from Colestid® tablets.

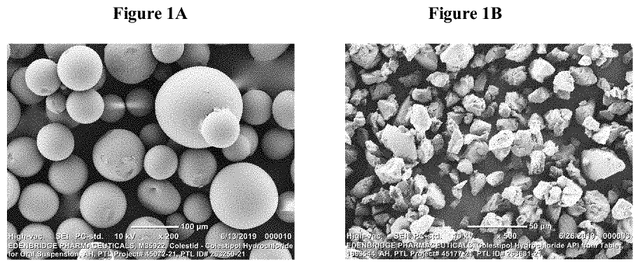

FIG. 2. Schematic representation of the conical rotor granulator in preferred configuration.

FIG. 3. Equilibrium moisture sorption/desorption of colestipol granules at 25° C.

FIG. 4. Monitoring of moisture uptake during rotor granulation process and subsequent drying of colestipol granules, measured using Loss on Drying (% LOD) and Near Infrared (NIR) probe.

### DETAILED DESCRIPTION OF THE INVENTION

The present invention contemplates a high dose finished pharmaceutical dosage form comprising colestipol hydrochloride in its commercially available form (i.e., beads) without the need for further milling or other particle size modification. The present invention does not require the use of the cutting mill described in U.S. Pat. No. 5,520,932.

The present invention may optionally include one or more pharmaceutically acceptable excipients. Exemplary pharmaceutically acceptable excipients for use in the present invention include binders/fillers during granulation, diluents/binders/fillers after granulation, disintegrants, glidants, and lubricants. A person of skill in the art would understand specific examples of each of the aforementioned classes of excipients.

Notwithstanding the above statement that a person of skill in the art would understand specific examples of each of the classes of binders/fillers during granulation, diluents/binders/fillers after granulation, disintegrants, glidants, and lubricants listed above, exemplary binders/fillers during granulation include, but are not limited to: polyvinyl pyrrolidone—PVP, K12, K17, K25, K30 and K90 grades (e.g., Kollidon 25, 30, 90 (BASF); Plasdone K12, K17, K25, K29/32 & K90 (Ashland)), copovidone PVA-PVP (e.g. Kollidon VA64 (BASF); Plasdone S630 (Ashland)), low molecular weight hypromellose grades (Methocel E3, E5, E6, E15LV, K3 (Dow); Pharmacoat 603, 605, 606, 615 (Shin-Etsu)), methylcellulose A15 (Methocel A15LV (Dow)), ethylcellulose (e.g., Ethocel (Dow); Aqualon EC (Ashland)), hydroxypropyl cellulose—HPC (Klucel ELF, EF, LF Grades (Ashland); Nisso HPC SL, L Grades (Nippon-Soda)), low substituted hydroxypropyl cellulose—L-HPC, sodium carboxymethylcellulose (Aqualon Grades 7L2P, 7LF (Ashland)), hydoxypropyl pea starch (Lycoat Grades (Rouquette)), polyethylene glycol—PEG (Carbowax PEG 1000, 3350, 4000, 8000 (Dow)), and pregelatinized starch (Starch 1500 (Colorcon); Lycatab PGS (Rouquette)). The binder may be introduced as a dry powder mixed into the API or dispensed continuously through another available port in the rotor granulator, with only water added as the binder through a second port. The preferred binder concentration is between 3-15% w/w and more preferably between 9-11% w/w.

Exemplary diluents/binders/fillers for use after granulation include starch, microcrystalline cellulose (e.g. Avicel Grades 101, 102, and 200 (Dupont); Emcocel 50M & 90M (JRS Pharma); Ceolus UF-702, UF-711, KG-802, KG-1000, PH 101, 102, 200, 301, 302, and F20JP (Asahi-Kasei)), hydroxypropyl cellulose, hypromellose, povidone, copovidone, lactose, mannitol, and dicalcium phosphate. A preferred diluent/binder/filler for use after granulation is microcrystalline cellulose (Ceolus KG-1000 (Asahi-Kasei) because its rodform particles fill in the gaps between the colestipol particles and allow for higher bulk density. In addition, Ceolus KG-1000 has excellent compressibility due to plastic deformation and produces tablets with relatively lower compression forces, compared to other microcrystalline cellulose grades. The final blend of the instant invention should exhibit a bulk density of at least 0.4 g/mL. Preferred embodiments of the instant invention exhibit a bulk density of greater than 0.5 g/mL. Even more preferred embodiments of the instant invention exhibit a bulk density of greater than 0.55 g/mL. As would be understood by a person of skill in the art, a higher bulk density which enables formation of a compact tablet.

Exemplary disintegrants include starch, croscarmellose sodium, sodium starch glycolate, cross-linked polyvinyl pyrrolidone, calcium carboxymethylcellulose, and low substituted hydroxypropyl cellulose (L-HPC). Exemplary glidants include colloidal silicas and talc. Exemplary lubricants include stearic acid, magnesium stearate, sodium stearyl fumarate, sodium lauryl sulfate, and polyethylene glycol (PEG 6000 & 8000).

A preferred embodiment of the present invention is a high dose formulation of colestipol hydrochloride comprising 1,000 mg of colestipol hydrochloride and a pharmaceutically acceptable diluent/binder/filler that is added after the granulation step of the present invention. This preferred embodiment may be made using a conical rotor granulator wherein the resulting granulation is compressed into tablets. The conical rotor granulator eliminates some shortcomings of traditional rotor technology by providing efficient mixing, flowability with concurrent tangential spraying, and densification of granules with low airflows. The spinning conical rotor contributes to the formation of spherical granules that are ideal for further processing. The granules of the preferred embodiment should exhibit bulk density of at least 0.40 g/mL. The tablets of this preferred embodiment may optionally be coated with one or more water-impermeable coatings to prevent moisture ingress into the tablet core (and thereby prevent issues with product stability). The dimensions of the preferred embodiment and its envelope density tablet dimensions are less than or equal to commercially available formulations of colestipol hydrochloride.

The high dose finished pharmaceutical dosage form of the instant invention is made using a granulation process. Traditional dry granulation of colestipol, alone or with various excipient mixtures, does not yield usable granules for compression because the beaded particles slip between the rollers. Wet granulation techniques utilizing mixers (high-shear and low-shear) are also not suitable because the wet process results in swollen API particles that, when dried, have a low bulk density. Although fluid bed granulators can form granules without over-wetting, the resulting particles had poor bulk density and compressibility. Further, the API shows an affinity for non-aqueous solvents, which cannot be removed from API, which renders the API unsafe for human consumption.

The direct blend compression process described U.S. patent application Ser. No. 13/467,448 for cholestyramine did not yield suitable granules of colestipol hydrochloride.

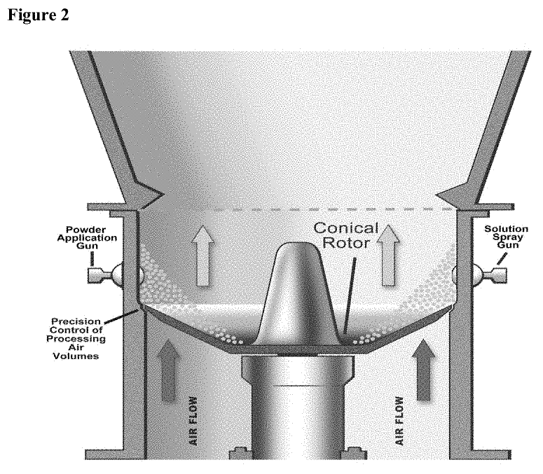

One skilled in the art would understand that rotor granulation can be performed using equipment (but not limited to) such as a centrifugal granulator (CF Granulator (e.g., Freund, Japan) or SpiraFlow® (e.g., Freund-Vector, Japan)) or rotor inserts placed inside conventional fluid-bed processors. Rotor inserts for fluid-bed processors can be flat rotors (e.g., Glatt GmbH, Binzen Germany) or conical rotors (e.g., Granurex® (Vector, Marion, Iowa, USA)). The Granurex® insert provides for a conical rotating disc inside a stationary container. The binder liquid is sprayed tangentially into the powder bed as the powder bed moves centrifugally. A separate drying air and low amount of fluidization air are introduced, to precisely control the moisture level in the powder bed. Moisture in the powder can be monitored by checking the Loss on Drying (% LOD) of the granules, sampled intermittently from the rotor chamber. Alternatively, the moisture level can be continuously monitored by introducing a Near Infrared (NIR) probe into the granulating chamber (e.g., RS-1000A, Innovative Technologies Group, Columbia, Md.). A representative configuration of the Granurex® conical rotor granulator is shown in FIG. 2.

The present invention identifies the correct balance between the need to reduce wetting and swelling of colestipol particles during manufacturing with the need to achieve granules with a suitable moisture content for tableting. The high dose finished pharmaceutical dosage form of the instant invention attains acceptable pharmaceutical stability, in vitro and in vivo bile acid binding performance, and shelf life.

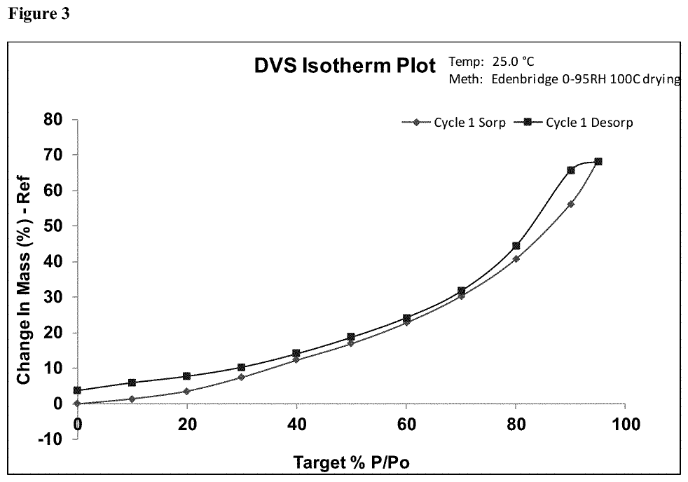

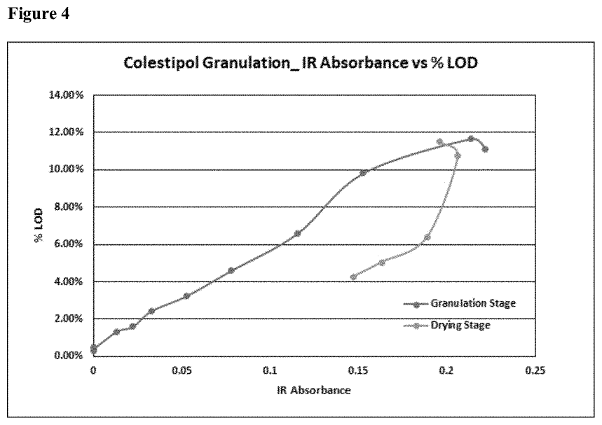

Colestipol hydrochloride drug substance and the granules show a high propensity for moisture uptake and swelling, as shown from the equilibrium moisture sorption/desorption graphs shown in FIG. 3, measured using dynamic vapor sorption. It was surprisingly discovered that if moisture content of the granulation is kept at 20% or less during the process, and the resulting granules are dried to a moisture content of 3-6%, acceptable tablets were created. In a preferred embodiment of the present invention, moisture content of the granulation is kept below 15% in the process, and the resulting granules are dried to a moisture content of 3-6%. This process control is carefully monitored as seen from FIG. 4, which shows a NIR trace of water throughout the process. The NIR trace was confirmed by a second method i.e. Loss on Drying (% LOD) on granule samples taken from inside the granulator. The final drug product should have a water content of not more than 7%. A preferred embodiment of the present invention has a drug product water content specification of 5-7%.

When manufacturing a finished pharmaceutical dosage form that is a tablet, optional coating(s) may be applied to prevent moisture ingress into tablets upon storage. Exemplary coating materials include but are not limited to cellulose acetate, ammoniomethacrylate copolymers, poly(ethylacrylate-methylmethacrylate), polyvinylacetate, methacrylate ester copolymers, cellulose acetate phthalate, polyvinyl acetate phthalate, methacrylic acid copolymers, cellulose acetate trimellitate, hydroxypropyl methylcellulose phthalate, cellulose acetate butyrate, hypromellose acetate succinate and ethycellulose. The coating is accomplished by dissolving the polymer, a plasticizer, and a glidant in an organic solvent or water:solvent mixtures and spraying onto the tablets. Exemplary plasticizers include but are not limited to phthalate esters (diethyl, dibutyl), dibutyl sebacate, citrate esters (triethyl, acetyl triethyl, acetyl tributyl), triacetin, castor oil, acetylated monoglycerides, and fractionated coconut oil. Organic solvents (e.g. ethanol, isopropyl alcohol, acetone, and methylene chloride) used in the coating do not adversely impact residual solvents in the product, due to instantaneous evaporation of the solvent during the coating process. A final polish layer may also be applied onto the tablets to smooth out the surface and impart an elegant appearance. The optional final polish layer also aids in uniform flow of tablets during a packaging operation. Exemplary coatings used for the final polish layer include cellulose derivatives such as hypromellose and hydroxypropyl cellulose or combinations thereof. Alternatively, the polishing can be attained by sprinkling a fine powder of wax at the end of coating process. Exemplary waxes used for such application include carnauba wax or beeswax.

The manufacturing process and techniques employed herein may be applicable to other high dose, insoluble polymeric drug substances such as, without limitation cholestyramine, colextran, colestilan, colesevelam, sevelemer hydrochloride, sevelemer carbonate, sodium polystyrene sulfonate, and patiromer.

The following examples are provided to help understand the invention but are not intended to limit the scope of the invention.

### EXAMPLE 1

The formula and process of one exemplary embodiment of the present invention are described below.

| Material | % w/w |
|---|---|
| Colestipol Hydrochloride | 81.0 |
| Copovidone | 10.0 |
| Microcrystalline Cellulose | 5.0 |
| Colloidal Silicon Dioxide | 0.6 |
| Cellulose Acetate Phthalate | 2.2 |
| Triacetin | 0.2 |
| Acetone* | |
| Opadry | 1.0 |
| Purified Water* | |
| | 100.0 |

*Not part of the finished pharmaceutical dosage form

Colestipol hydrochloride drug substance is granulated in a rotor granulator using an aqueous solution of copovid one to achieve uniform granules with an optimal moisture content (moisture content of the granulation is kept below 15% during the process, and the resulting granules are dried to a moisture content of 3-6%), which exhibit a bulk density of at least 0.4 g/mL. The granules are screened through a #30 mesh screen and the larger granules milled using an attrition mill, if necessary. The screened granules are blended with microcrystalline cellulose and colloidal silicon dioxide. The blend is compressed using a tablet press (Kilian). The core tablets are coated with a solution of cellulose acetate phthalate and triacetin in acetone followed by a polish layer consisting of Opadry. The finished pharmaceutical dosage form exhibits dimensions that are comparable (or less than) to Colestid® tablets and cholate binding efficiency within 1.1-1.6 mEq/g. The tablets do not show any moisture uptake upon storage and disintegrate within 20 min.

### EXAMPLE 2

The formula and process of another exemplary embodiment of the present invention are described below.

| Material | % w/w |
|---|---|
| Colestipol Hydrochloride | 82.0 |
| Povidone | 9.0 |
| Microcrystalline Cellulose 20 μm | 4.0 |
| Sodium Starch Glycolate | 1.0 |
| Colloidal Silicon Dioxide | 0.4 |
| Polyethylene Glycol 6000 | 0.4 |
| Cellulose Acetate | 2.0 |
| Triacetin | 0.2 |
| Acetone* | |
| Opadry | 1.0 |
| Purified Water* | |
| | 100.0 |

*Not part of the finished pharmaceutical dosage form

Colestipol hydrochloride drug substance is granulated in a rotor granulator using an aqueous solution of povidone, to achieve uniform granules with an optimal moisture content (moisture content of the granulation is kept below 15% during the process, and the resulting granules are dried to a moisture content of 3-6%), which exhibit a bulk density of at least 0.4 g/mL. The granules are screened through a #30 mesh screen and the larger granules milled using an attrition mill, if necessary. The screened granules are blended with microcrystalline cellulose, sodium starch glycolate, and colloidal silicon dioxide. PEG 6000 is added and further blended. The blend is compressed using a tablet press (Kilian). The core tablets are coated with a solution of cellulose acetate and triacetin in acetone followed by a polish layer consisting of Opadry. The finished pharmaceutical dosage form exhibits dimensions that are comparable (or less than) to Colestid® tablets and cholate binding efficiency within 1.1-1.6 mEq/g. The tablets do not show any moisture uptake upon storage and disintegrate within 20 min.

### EXAMPLE 3

The formula and process of another exemplary embodiment of the present invention are described below.

| Material | % w/w |
|---|---|
| Colestipol Hydrochloride | 82.0 |
| Hydroxypropyl cellulose (HPC-SL) | 9.4 |
| Microcrystalline Cellulose 20 μm | 4.0 |
| L-HPC | 1.0 |
| Colloidal Silicon Dioxide | 0.4 |
| Cellulose Acetate Phthalate | 3.0 |
| Triacetin | 0.2 |
| Acetone* | |
| Carnauba Wax | Trace |
| | 100.0 |

*Not part of the finished pharmaceutical dosage form

Colestipol hydrochloride drug substance is granulated in a rotor processor using an aqueous solution of hydroxypropyl cellulose, to achieve uniform granules with an optimal moisture content (moisture content of the granulation is kept below 15% during the process, and the resulting granules are dried to a moisture content of 3-6%), which exhibit a bulk density of at least 0.4 g/mL. The granules are screened through a #30 mesh screen and the larger granules milled using an attrition mill, if necessary. The screened granules are blended with microcrystalline cellulose, L-HPC, and colloidal silicon dioxide. The blend is compressed using a tablet press (Kilian). The core tablets are coated with a solution of cellulose acetate phthalate and triacetin in acetone. At the end of coating carnauba wax is sprinkled onto tablets in the coating pan and tumbled further. The finished pharmaceutical dosage form exhibits dimensions that are comparable (or less than) to Colestid® tablets and cholate binding efficiency within 1.1-1.6 mEq/g. The tablets do not show any moisture uptake upon storage and disintegrate within 20 min.

What is claimed is:

1. A high dose finished pharmaceutical dosage form comprising colestipol hydrochloride as an active pharmaceutical ingredient wherein the high dose finished pharmaceutical dosage form has a water content of not more than 7%.

2. The high dose finished pharmaceutical dosage form of claim 1 wherein the high dose finished pharmaceutical dosage form comprises colestipol hydrochloride as an active pharmaceutical ingredient and at least one pharmaceutically acceptable excipient.

3. The high dose finished pharmaceutical dosage form of claim 1 wherein the high dose finished pharmaceutical dosage form comprises colestipol hydrochloride as an active pharmaceutical ingredient and at least one pharmaceutically acceptable excipient wherein the at least one pharmaceutically acceptable excipient is a binder.

4. The high dose finished pharmaceutical dosage form of claim 1 wherein the high dose finished pharmaceutical dosage form comprises colestipol hydrochloride as an active pharmaceutical ingredient and at least one pharmaceutically acceptable excipient wherein the at least one pharmaceutically acceptable excipient is a binder that is 9-11 percent w/w of the high dose finished pharmaceutical dosage form.

5. The high dose finished pharmaceutical dosage form of claim 1 wherein the high dose finished pharmaceutical dosage form comprises colestipol hydrochloride as an active pharmaceutical ingredient and copovid one wherein the copovid one is 9-11 percent w/w of the high dose finished pharmaceutical dosage form.

6. The high dose finished pharmaceutical dosage form of claim 1 wherein the high dose finished pharmaceutical dosage form comprises colestipol hydrochloride as an active pharmaceutical ingredient and at least one pharmaceutically acceptable excipient wherein the at least one pharmaceutically acceptable excipient is microcrystalline cellulose with rod-shaped particles.

7. A process for manufacturing the high dose finished pharmaceutical dosage form of claim 1 comprising
preparing a granulation of the colestipol hydrochloride in a rotor granulator to form granules of colestipol hydrochloride wherein the granulation has a moisture content of 20% or less and
drying the granules of colestipol hydrochloride to a moisture content of 3-6%.
